(19) United States
(12) Patent Application Publication
(10) Pub. No.: US 2019/0183798 A1
(43) Pub. Date: Jun. 20, 2019

(54) METHOD FOR TREATING TUMOR

(71) Applicant: Johnpro Biotech Inc., New Taipei City (TW)

(72) Inventors: Kwan-Hwa CHI, Taipei City (TW); Yu-Shan WANG, New Taipei City (TW); Hsin-Chien CHIANG, New Taipei City (TW); Cheng-Chung HUANG, Nantou County (TW)

(73) Assignee: Johnpro Biotech Inc., New Taipei City (TW)

(21) Appl. No.: 16/309,101

(22) PCT Filed: Jun. 17, 2016
(86) PCT No.: PCT/CN2016/086200

§ 371 Date: Dec. 11, 2018

(51) Int. Cl. A61K 9/127; A61N 5/10; A61N 1/40

(52) U.S. Cl. CPC A61K 9/127; A61K 9/0019; A61N 1/403; A61N 5/10

(57) ABSTRACT

A method of treating a subject having or suspected of having a solid tumor, comprises administering to the subject an effective amount of a liposome-encapsulated drug, and applying an amplitude-modulated (AM) radiofrequency radiation to the solid tumor.

### BACKGROUND OF THE INVENTION

### 1. Field of the Invention

The present disclosure in general relates to the field of tumor treatment. More particularly, the present disclosure relates to a method for treating tumors by use of liposomes and the amplitude-modulated (AM) radiofrequency radiation.

### 2. Description of Related Art

Tumor is one of the most common lethal diseases in the world, with 14 million new cases diagnosed annually and is also the leading cause of deaths worldwide, causing 8.2 million deaths annually as World Health Organization (WHO) reported in the World Cancer Report 2014. Nowadays, different treatments have been developed to treat tumors, such as surgery, chemotherapy, radiotherapy, anti-angiogenic therapy, and immunotherapy. However, all these therapies remain insufficient survival benefit.

A liposome is a spherical vesicle having at least one lipid bilayer. The liposome can be used as a vehicle for encapsulating and delivering the nutrients or pharmaceutical drugs to desired sites. Generally, the lipid bilayer can fuse with other bilayers (such as the cell membrane), thereby introduces the liposome-encapsulated nutrients or drugs into cells of the target sites. As of 2012, 13 drugs with liposomal delivery systems have been approved and five additional liposomal drugs are evaluated in clinical trials, including the treatment of infectious diseases and/or tumors.

In recent years, several strategies have being adopted for improving the efficacy of liposome on the treatment of tumors, such as (1) increasing the targeting specificity to tumor cells by conjugating tumor-specific moieties to the liposomal surface; for example, it has been reported that the conjugation of epidermal growth factor receptor (EGFR) to the liposomal surface enables the liposome specifically target to EGFR-overexpressed tumor cells; (2) increasing the targeting specificity to tumor microenvironment by conjugating the antibody fragment against angiogenesis-associated factors (e.g., endothelial growth factor (VEGF), cell adhesion molecule (CAM), matrix metalloprotease (MMP), and integrin) to the liposomal surface; the thus-produced liposome may efficiently targeting to the endothelial cells or fibroblasts that involves in the angiogenesis, tumor growth, and tumor metastasis; (3) increasing the drug release in the tumor regions; for example, the use of stimuli-sensitive liposome that releases drugs in response to the internal or external stimuli (e.g., pH, temperature, enzyme, light, ultrasound, and magnetics); (4) increasing the stability of liposome; it is known that Inclusion of polyethylene glycol (PEG)-modified lipids contributes to interfering the liposome recognition by phagocytic cells; further, cholesterol, tocopherol or other membrane active antioxidant incorporation may also increase the stability of liposome. However, each of the strategies listed above has its limitation, such as high-cost, low efficiency, and/or time-consuming.

In view of the foregoing, there exists in the related art a need of an improved method that enhances the therapeutic efficacy of a liposomal drug, which in term will improve treatment efficacy of a subject having or suspected of having tumors.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present disclosure aims at providing a means for improving the therapeutic efficacy of liposome on treating tumors. As embodied and broadly described herein, one aspect of the disclosure is directed to a method of treating a subject having or suspected of having a solid tumor. The method comprises the steps of,

(1) administering to the subject an effective amount of a liposome-encapsulated therapeutic agent; and

(2) applying an AM radiofrequency radiation to the solid tumor so as to enhance the absorption of the liposome-encapsulated therapeutic agent into the solid tumor.

According to some embodiments, the therapeutic agent encapsulated in the liposome is selected from the group consisting of, an anti-tumor agent, an anti-inflammatory agent, an anti-angiogenic agent, an immunomodulatory agent, and a radionuclide.

According to one embodiment, the therapeutic agent is the anti-tumor agent, which is selected from the group consisting of bleomycin, epirubicin, estramustine, etoposide, 5-fluorouracil, doxorubicin, mitomycin C, cisplatin, paclitaxel, camptothecin, vincristine, vinblastine, methotrexate, mitoxantrone, thalidomide and squalamine. In one working example, the therapeutic agent is bleomycin, doxorubicin, mitomycin C, cisplatin or paclitaxel. Preferably, the therapeutic agent encapsulated in the liposome has a concentration of about 5-800 μg/ml.

Structurally, the present liposome has a diameter ranging from about 50 nm to about 5,000 nm.

In general, the present liposome-encapsulated therapeutic agent can be administered to the subject via an oral, enteral, nasal, topical, transmucosal, intramuscular, intravenous, intraarterial, subcutaneous, intraperitoneal, or intratumoral route.

According to the embodiments of the present disclosure, the radiofrequency of the AM radiofrequency radiation is about 1-50 MHz, and the power of the AM radiofrequency radiation is about 0.5-300 watts.

According to the certain embodiments, the solid tumor is melanoma, esophageal carcinoma, gastric carcinoma, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, cervical cancer, or head and neck squamous cell carcinoma.

The subject treatable by the present method is a mammal, for example, a human, a monkey, a chimpanzee, a mouse, a rat, a house, a rabbit, a pig, a sheep, a goat, a cat and a dog. Preferably, the subject is a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A-1B are the results of flow cytometry assay and confocal microscopy that respectively depict the level (FIG. 1A) and retention time (FIG. 1B) of doxorubicin in the cells treated with specified treatment.

FIGS. 2A-2C are the histograms that respectively depict the level of dextran (FIG. 2A), CT-B (FIG. 2B), and transferrin (FIG. 2C) in the cells treated with specified treatment.

FIG. 3 is a histogram that depicts the level of doxorubicin in the tumors of mice respectively treated with specified treatments.

In accordance with common practice, the various described features/elements are not drawn to scale but instead are drawn to best illustrate specific features/elements relevant to the present invention. Also, like reference numerals and designations in the various drawings are used to indicate like elements/parts.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “liposome” as used herein refers to an artificially and/or naturally prepared vesicle composed of at least one lipid bilayer. A liposome may be in a form of unilamellar vesicle (i.e., the vesicle comprised of a single lipid bilayer) or multilamellar vesicle (i.e., the vesicle comprised of several lipids bilayers).

A used herein, the term “therapeutic agent” refers to a compound, molecule, or treatment that elicits a biological response from any host, animal, vertebrate, or invertebrate, including for example fish, mammals, amphibians, reptiles, birds, and humans. Examples of therapeutic agents include, but are not limited to, anti-tumor agent, anti-inflammatory agent, anti-angiogenic agent, immunomodulatory agent, and radionuclides.

As used herein, the term “anti-tumor agent” refers to a molecule that is capable of inhibiting the proliferation of or killing cells of tumor tissues. More specifically, the anti-tumor agent may (1) reduce the proliferation of tumor cells, (2) inhibit or prevent the migration of tumor cells, (3) inhibit the colony formation of tumor cells due to the anchorage-independent growth, (4) induce the necrosis of the tumor cells, and/or (5) induce the apoptosis of the tumor cells.

As used herein, the term “anti-inflammatory agent” refers to a molecule that is capable of counteracting, preventing, and/or reducing tissue inflammation caused by infection, disease, auto-antibody, and/or trauma.

As used herein, the term “anti-angiogenic agent” is used interchangeably with the term “anti-angiogenic agent” or “angiogenesis inhibitor”. More specifically, the term “anti-angiogenic agent” herein refers to a molecule that has the ability to (1) inhibit the proliferation or the migration of endothelial cells, (2) kill the proliferating endothelial cells, and/or (3) inhibit the formation of new blood vessels in a tissue.

The term “immunomodulatory agent” herein refers to a molecule that modulates one or more of the components (e.g., immune cells, or subcellular factors, genes regulating immune components, cytokines, chemokines or such molecules) of a host's immune system. The immunomodulatory agent can be an immunosuppressive agent or an immunostimulatory agent.

As used herein, the term “radionuclide” refers to an atom with an unstable nucleus, which undergoes radioactive decay and emits gamma rays and/or subatomic particles. The example of radionuclide includes, but is not limited to, ⁹⁰Y, ¹¹¹In, ⁶⁷Cu, ⁷⁷Lu, ¹⁷⁷Lu, and ⁹⁹Tc.

The term “radiofrequency” (RF) herein refers to the electromagnetic waves in the range of from about 3 KHz to about 300 GHz.

The term “solid tumor” herein refers to an abnormal mass of tissue that is typically devoid of cysts or liquid areas. Solid tumors may be benign (not cancer), or malignant (cancer). Different types of solid tumors are named for the type of cells that form them. Examples of solid tumors are sarcomas, carcinomas, and lymphomas.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/Kg). Alternatively, the effective amount can be expressed in the concentration of the active component (e.g., the liposome-encapsulated therapeutic agent of the present disclosure), such as molar concentration, mass concentration, volume concentration, molality, mole fraction, mass fraction and mixing ratio. Specifically, the term “therapeutically effective amount” used in connection with liposome-encapsulated therapeutic agent described herein refers to the quantity of liposome-encapsulated therapeutic agent, which is sufficient to alleviate or ameliorate the symptoms associated with the tumors in the subject. Persons having ordinary skills could calculate the human equivalent dose (HED) for the medicament (such as the present fusion protein) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “subject” refers to a mammal including the human species that is treatable with methods of the present invention. The term “subject” is intended to refer to both the male and female gender unless one gender is specifically indicated.

The objective of the present disclosure aims at providing a method that improves the absorption of a liposome-encapsulated therapeutic agent into a treatment site e.g., the site of a tumor. Accordingly, one aspect of the present disclosure is directed to a method of treating a subject having or suspected of having a solid tumor. The present method includes at least two steps of,

(1) administering to the subject an effective amount of a liposome-encapsulated therapeutic agent; and

(2) applying an AM radiofrequency radiation to the solid tumor so as to enhance the absorption of the liposome-encapsulated therapeutic agent into the solid tumor.

In the step (1), a liposome-encapsulated therapeutic agent is administered to the subject via any suitable route. According to the embodiments of the present disclosure, the liposome-encapsulated therapeutic agent comprises a liposome and a therapeutic agent encapsulated in the liposome.

The liposome may be made of natural and/or synthetic lipids. Natural lipids include, for example, Lipid A (e.g., Detoxified Lipid A), cholesterol, sphingolipids (e.g., spingosine and derivatives such as d-erythro-sphingosine, sphingomyelin, ceramides, cerebrosides, brain sulfatides), gangliosides, sphingosine derivatives (e.g., glucosylceramide), phytosphingosine and derivatives (e.g., phytosphingosine, d-ribo-phytosphingosine-1-phosphate, N-acyl phytosphingosine C2, N-acyl phytosphingosine C8, and N-acyl phytosphingosine C18), choline (e.g., phosphatidylcholine and platelet-activation factor), ethanolamine (e.g., phosphatidylethanolamine), glycerol (e.g., phosphatidyl-dl-glycerol), inositol (e.g., phosphatidylinositol and phosphatidylinositol), serine (e.g., phosphatidylserine (sodium salt)), cardiolipin, phosphatidic acid, egg derivatives, lyso (mono acyl) derivatives (lysophosphatides), hydrogenated phospholipids, lipid tissue extracts (e.g., brain, heart, liver, egg, soy, and Escherichia coli,), and fatty acid content of tissue derived phosolipids (e.g., phosphatidylcholine, phosphatidylethanolamine). The examples of the synthetic lipid include, but are not limited to, phosphaditylcholine, phosphatidylethanolamine, phosphatidylserine, phosphatidylinositol, phosphatidic acid, phosphatidylglycerol, cardiolipin, diacylglycerides, cholesterol, polyethylene glycol (PEG, including PEG-350, PEG-400, PEG-450, PEG-500, PEG-550, PEG-750, PEG-800, PEG-1000, PEG-2000, PEG-3000, and PEG-5000) lipid, functionalized lipid for conjugation, phospholipid with multifarious headgroups, lipid for pH sensitive liposomes, metal chelating lipid, antigenic phospholipid, doxyl lipid, fluorescent lipid, lyso phospholipid, alkyl phosphocholine, oxidized lipid, biotinylated, ether lipid (including diether Ipids, alkyl phosphocholine, and O-alkyl diacylphosphatidylcholinium), plasmologen lipid, diphytanoyl phospholipid, polymerizable lipid, brominated phospholipid, fluorinated phospholipid, deuterated lipid, doxyl lipid, fluorescent lipid, bioactive glycerol-based lipid (e.g., platelet activation factor lipid and second messenger lipid), and lipid metabolic intermediate. Depending on the lipid selected, the produced liposome may be cationic, anionic, and/or neutral. According to one embodiment of the present disclosure, the present liposome is made of distearoylphosphatidylcholine (DSPC), cholesterol (CH), or polyethylene glycol 2000-distearoylphosphatidylethanolamine(PEG-2000-DSPE).

Further, a functional molecule may be incorporated into and/or conjugated to the surface of the liposome described above. For example, a tumor-associated ligand or receptor, or a tumor-specific antibody can be conjugated to the liposomal surface so as to increase the specificity (either to the tumor cell or to the tumor-supportive cell (e.g., endothelial cell and fibroblast)) of the present liposome. Additionally and/or alternatively, the complement C3b can be conjugated to the liposomal surface to inhibit the recognition by phagocytic complement receptors and thus prolong the half-life of the present liposome in vivo.

As to the therapeutic agent, it can be an anti-tumor agent, an anti-inflammatory agent, an anti-angiogenic agent, an immunomodulatory agent, or a radionuclide. According to one preferred example, the therapeutic agent is the anti-tumor agent; non-limiting anti-tumor agent includes, but is not limited to, bleomycin, epirubicin, estramustine, etoposide, 5-fluorouracil, doxorubicin, mitomycin C, cisplatin, paclitaxel, camptothecin, vincristine, vinblastine, methotrexate, mitoxantrone, thalidomide and squalamine. Preferably, the therapeutic agent is bleomycin, doxorubicin, mitomycin C, cisplatin or paclitaxel. More preferably, the therapeutic agent is bleomycin.

Alternatively, the therapeutic agent is the anti-inflammatory agent. The anti-inflammatory agent can be a steroidal anti-inflammatory drug (SAID) or a non-steroidal anti-inflammatory drug (NSAID). Suitable example of the anti-inflammatory agent includes, but is not limited to, aspirin, ibuprofen, naproxen, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

Optionally, the therapeutic agent can be the anti-angiogenic agent, which is selected from the group consisting of endostatin, angiostatin, thrombospondin-1 (TSP-1), calreticulin (CRT), tumstatin, vasoinhibins, vasculostatin, arresten, canstatin, endoglin, restin, maspin, pigment epithelium-derived factor (PEDF), endorepellin, platelet factor 4 TNP-470, Zoledronic acid and antibody (such as Bevacizumab, Ranibizumab, Sunitinib and Pegaptanib).

As to the immunomodulatory agent, it can be antibiotic (such as clindamycin, aminoglycosides, erythromycin and β-lactam antibiotics), statin (such as atorvastatin, cerivastatin, fluvastatin, lovastatin, mevastatin, pitavastatin, pravastatin, rosuvastatin and simvastatin), interferon (IFN, including IFN-α, IFN-β and IFN-γ), glatiramer acetate, methotrexate, BG12, fingolimod, mixoxantrone, laquinimod, teriflunomide, atorvastatin, thalidomide, lenalidomide, pomalidomide, apremilast, steroid, or the combination thereof.

Alternatively, the therapeutic agent encapsulated in the present liposome is a radionuclide selected from the group consisting of ⁹⁰Y, ¹¹¹In, ⁶⁷Cu, ⁷⁷Lu, ¹⁷⁷Lu, and ⁹⁹Tc.

As would be appreciated, the present liposome may comprise more than one therapeutic agent so as to enhance the tumor treating effect. For example, for the purpose of treating the hepatitis virus-induced tumor (such as hepatitis B virus-induced hepatocellular carcinoma, and hepatitis C virus-induced hepatocellular carcinoma) that is associated with and/or resulted from the inflammation of the liver, the present liposome may further encapsulate an anti-inflammatory agent, in addition to the anti-tumor agent; in this way, the anti-tumor agent may suppress t the growth and/or result in the death (e.g., necrosis or apoptosis) of the tumor cell, while the anti-inflammatory agent reduces the level of the inflammatory factors so as to alleviate the inflammation inside and/or around the tumor microenvironment. Alternatively, the present liposome may further include an anti-angiogenic agent, in addition to the anti-tumor agent; thus, the anti-tumor agent may suppresss the growth and/or results in the death (e.g., necrosis or apoptosis) of the tumor cell, while the anti-angiogenic agent blocks the angiogenesis process that supports the growth of the tumor cell. The skilled artisan may choose and combine different numbers and/or kinds of therapeutic agents to produce the present liposome in accordance with the requirement of application. As would be appreciated, when the present liposome comprises more than one therapeutic agent, the ratio of these agents may vary with the desired effect.

The liposome can be prepared by use of any known methods, such as a thin-membrane hydration method, an ultrasonification method, an ethanol injection method, an ether injection method, a reverse-phase evaporation method, a surfactant method, a freezing/thawing method, and a thin-membrane hydration-ultrasonic method.

According to some embodiments, the therapeutic agent encapsulated in the liposome has a concentration about 5-800 ug/ml; that is, the concentration may be 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, or 800 μg/ml. According to one preferred example, the concentration of the therapeutic agent is about 25-150 μg/ml.

The particle diameter of the liposome can be modulated by known methods; for example, an extrusion method, a French press method, and a homogenization method. According to the embodiments of the present disclosure, the liposome encapsulating the therapeutic agent has a diameter ranging from about 50 nm to about 5,000 nm; for example, 80, 90, 100, 200, 300, 400, 500, 600, 700, 800, 900, 1,000, 1,500, 2,000, 2,500, 3,000, 3,500, 4,000, 4,500, or 5,000 nm. Preferably, the diameter of the liposome encapsulating the therapeutic agent is about 80 nm to 5,000 nm.

The liposome-encapsulated therapeutic agent may be formulated with a suitable carrier, thereby is administered in the form of an aqueous solution, an ointment, a gel, a paste, a lotion, a sponge, a spray, or soft gelatin capsules. The liposome-encapsulated therapeutic agent may be administered in a paste or gel which is placed in a soft gelatin capsule. In a preferred embodiment, the liposome-encapsulated therapeutic agent are administered in an aqueous solution.

Optionally, the aqueous solution comprising the present liposome-encapsulated therapeutic agent may further comprise one or more pharmaceutical additives selected from buffer, stabilizer, isotonic agent, pH adjustor, solubilizing agent, thickener, dispersant, preservative, and the like, if necessary.

Non-limiting example of the buffer includes boric acid, phosphoric acid, acetic acid, carbonic acid, citric acid, and their equivalent substances. The equivalent substances to phosphoric acid, acetic acid, carbonic acid, and citric acid mean compounds which generate phosphate ion, acetate ion, carbonate ion, and citrate ion on dissolution in water.

Non-limiting example of the stabilizer includes tocopherol, butylhydroxyanisole, butylhydroxytoluene, ethylenediaminetetraacetic acid (EDTA), and cysteine.

The example of the isotonic agent includes, but is not limited to, D-mannitol, D-sorbitol, D-xylitol, glycerin, glycerol, glucose, sorbitol, mannitol, maltose, sucrose, propylene glycol, and electrolyte (such as sodium chloride and potassium chloride).

The pH adjustor includes, for example, hydrochloric acid, citric acid, phosphoric acid, acetic acid, sodium hydroxide, potassium hydroxide, boric acid, borax, sodium carbonate, sodium bicarbonate etc. Preferably, the aqueous solution comprising the present liposome-encapsulated therapeutic agent has a pH of 7 to 9; more preferably, a pH of 7 to 7.4.

Non-limiting example of the solubilizing agent includes polysorbate 80, polyoxyethylene hardened castor oil 60, macrogol 4000, polyethylene glycol, and propylene glycol.

The example of the thickener and dispersant include, but is not limited to, macromolecule of cellulose type (such as hydroxypropylmethylcellulose, hydroxypropylcellulose), sodium alginate, polyvinyl alcohol, carboxyvinylpolymer, and polyvinyl pyrrolidone.

The preservative as an therapeutic agent against fungi and bacteria includes, but is not limited to, benzalkonium chloride, benzethonium chloride, chlorhexidine, parabens (such as methyl paraben and ethyl paraben), and thimerosal.

In general, the liposome-encapsulated therapeutic agent is administered to the subject via an oral, enteral, nasal, topical, transmucosal, intramuscular, intravenous, intraarterial, subcutaneous, intraperitoneal, or intratumoral route.

As known to the skilled artisan, the two most common types of modulation used in radiofrequency are amplitude modulation (AM) and frequency modulation (FM). In the AM transmission, the carrier wave is constant in frequency and varies in amplitude (strength); in contrast, the carrier is constant in amplitude and varies in frequency in the FM transmission. The inventors of the present disclosure unexpectedly discovered that the AM radiofrequency radiation can improve the absorption of the liposome-encapsulated therapeutic agent by the organ and/or tissue (e.g., solid tumor). Accordingly, in the step (2), the AM radiofrequency radiation is applied to the solid tumor to enhance the therapeutic efficacy of the liposome-encapsulated therapeutic agent administered in the step (1).

According to the embodiments of the present disclosure, compared with the hyperthermia treatment (i.e., 37° C. or 42° C. treatment) and the conventional radiofrequency radiation (i.e., thermotron RF-8), AM radiofrequency radiation may enhance the uptake of the present liposome-encapsulated therapeutic agent into the tumor cells in a more efficient manner.

According to some embodiments of the present disclosure, the radiofrequency of the AM radiofrequency radiation plays a key role in the present method, in which the uptake of the liposome-encapsulated therapeutic agent is enhanced by the application of AM radiofrequency radiation to the tumor site. According to these embodiments, the radiofrequency of the AM radiofrequency radiation that resulted in the enhanced absorption of the present liposome-encapsulated therapeutic agent is about 1-50 MHz; such as 1, 2, 3, 4, 5, 6, 7, 8, 9, 10, 15, 20, 25, 30, 35, 40, 45, or 50 MHz. Preferably, the radiofrequency of the AM radiofrequency radiation is about 5-45 MHz. More preferably, the radiofrequency is about 8-20 MHz.

As to the power of the AM radiofrequency radiation, it depends on the subject that received the treatment. Basically, the power of the AM radiofrequency radiation is about 0.5-300 watts; for example, 0.5, 0.6, 0.7, 0.8, 0.9, 1.0, 2.0, 3.0, 4.0, 5.0, 6.0, 7.0, 8.0, 9.0, 10.0, 20.0, 30.0, 40.0, 50.0, 60.0, 70.0, 80.0, 90.0, 100.0, 110.0, 120.0, 130.0, 140.0, 150.0, 160.0, 170.0, 180.0, 190.0, 200.0, 210.0, 220.0, 230.0, 240.0, 250.0, 260.0, 270.0, 280.0, 290.0, or 300.0. Preferably, the power of the AM radiofrequency radiation is about 0.5-150 watts. According to one embodiment, the AM radiofrequency radiation is applied in vitro to enhance the absorption of the present liposome-encapsulated therapeutic agent by tumor cells), and the power of the AM radiofrequency radiation is about 5-20 watts. According to another embodiment, the AM radiofrequency radiation is applied in vivo to enhance the uptake and/or absorption of the present liposome-encapsulated therapeutic agent by a mammal (including human, monkey, chimpanzee, mouse, rat, house, rabbit, pig, sheep, goat, cat and dog etc.), and the power of the AM radiofrequency radiation is preferably in the range of about 25-150 watts.

In the present invention, pink noise (also known as 1/f noise or flicker noise) modulation is preferable, but the pink noise spectrum is not necessary in all cases. The modulated radiofrequency radiation has the advantages of,
(1) the energy uptake in non-pink noise areas will be higher than in pink noise areas; for most of the healthy physiological effects have pink-noise signal exchange, while the malignant tissue does not, the pink noise modulation is selectively damaging to malignant tissue; and
(2) the modulation increases the electric field gradient in the extracellular liquid and the amplitude (electric field) in the area; this order makes the social signal available (apoptosis induction) and “glues” the dividing cells into a fixed position (metastasis block).

Depending on the desired effect, the AM radiofrequency radiation can be applied to the solid tumor before, during, or after the administration of the present liposome-encapsulated therapeutic agent.

According to some embodiments of the present disclosure, the temperature of the solid tumor is maintained at less than 42° C. during the application of the AM radiofrequency radiation. In one example, the temperature of the solid tumor is maintained at 37-37.5° C. in the step (2) of the present method. In another example, the temperature of the solid is maintained at 41.5-42° C. in the step (2) of the present method.

According to certain embodiments of the present disclosure, the subject is a mammal, such as human, monkey, chimpanzee, mouse, rat, house, rabbit, pig, sheep, goat, cat and dog. Preferably, the subject is a human.

According to the embodiment, the solid tumor treatable by the present method is any of melanoma, esophageal carcinoma, gastric carcinoma, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, cervical cancer, or head and neck squamous cell carcinoma.

As would be appreciated, the present method can be applied to the subject having or suspected of having a solid tumor, alone or in combination with other conventional therapies (such as surgery, chemotherapy, radiotherapy, anti-angiogenic therapy, and immunotherapy) that have some beneficial effects on the treatment of tumors. Depending on the therapeutic purpose, the present method can be applied to the subject before, during, or after the administration of the conventional therapy.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

Materials and Methods

Cell Culture

The HepG2 cell (ATCC® HB-8065™) was purchased from American Type Culture Collection (ATCC) and cultured in Eagle's Minimum Essential Medium (DMEM) containing 10% heat-inactivated fetal bovine serum (FBS). The CT26 cell, a murine colon carcinoma cell line derived from a BALB/c mouse, was purchased from the Culture Collection and Research Center (Hsinchu, Taiwan), where fresh batches are thawed every year. The CT26 cell was maintained in DMEM supplemented with 10% FBS, 100 ng/ml of streptomycin, and 100 U/ml of penicillin (Invitrogen). All the cells were incubated in a 37° C. incubator containing a humidified atmosphere of 5% CO₂.

Preparation of Liposome-Encapsulated Doxorubicin (Lipo-Dox)

The PLD used in this study, Lipo-Dox™ (20 mg/10 mL), was purchased from TTY Biopharm (Taipei, Taiwan).

Treatment of Cell by Drug

To investigate the effect of specified treatments on the absorption of lipo-dox, HepG2 cells were randomly assigned to 5 groups: (1) untreated cells, which served as the control group; (2) 37° C. treatment group, in which the cells were treated with 20-25 μg/ml lipo-dox followed by incubating at the temperature of 37° for 30 minutes; (3) 42° C. treatment group, in which the cells were treated with 20-25 μg/ml lipo-dox followed by incubating at the temperature of 42° C. for 30 minute; (4) mEHT treatment group, in which the cells were treated with 20-25 μg/ml lipo-dox and then exposed to modulatedelectro-hyperthermia (mEHT) for 30 minute; and (5) RF-8 treatment treatment group, in which the cells were treated with 20-25 μg/ml lipo-dox and then exposed to thermotron RF-8 (Thermotron RF-8; Yamamoto Vinita Co., Osaka, Japan) for 30 minutes. The detail condition was summarized in Table 1.

TABLE 1
Condition for cell treatment

| | | 37° C. | 42° C. | mEHT | RF-8 |
|---|---|---|---|---|---|
| Group | Untreatment | treatment | treatment | treatment | treatment |
| Drug | None | Lipo-dox | Lipo-dox | Lipo-dox | Lipo-dox |
| Temperature | 30 min | 30 min | 30 min | 30 min | 30 min |
| Temperature | 37° C. | 37° C. | 42° C. | 37 or 42° C. | 42° C. |
| Radiofrequency | None | None | None | 13.56 MHz | 8 MHz |
| Modulation | None | None | None | Yes | None |
| Watt | 0 | 0 | 1.2-1.4 | 10-12 | 16-19 |
| | | | (KVA)* | | |
| Increment | 1 | 5.09 | 6.57 | 17.46 | 7.54 |
| (fold)** | | | | | |

*KVA: kilovolt-amps.
**compared with the untreatment group.

With the similar procedure, HepG2 cells were divided into 4 groups in the experiments investigating the effect of specified treatments on the absorption of dextran, CT-B, or transferrin, including: (1) untreated cell, which serves as the control group; (2) 37° C. treatment, in which the cells were treated with tested drug (1 mg/ml of dextran, 2 μg/ml of CT-B, or 5 μg/ml of transferrin) followed by being incubated at the temperature of 37° for 30 minutes; (3) 42° C. treatment, in which the cells were treated with tested drug (1 mg/ml of dextran, 2 μg/ml of CT-B, or 5 μg/ml of transferrin) followed by being incubated at the temperature of 42° C. for 30 minute; (4) mEHT treatment, in which the cells were treated with tested drug (1 mg/ml of dextran, 2 μg/ml of CT-B, or 5 μg/ml of transferrin) and then exposed to modulatedelectro-hyperthermia (mEHT) for 30 minute.

In the treatment of wortmannin, an inhibitor of macropinocytosis pathway, the HepG2 cells were treated with phosphate-buffered saline (PBS) or wortmannin at 37° C. for 15 minutes. After the administration of dextran, the cells were applied with 37° C. treatment, 42° C. treatment or mEHT treatment for 1 hour.

Confocal Microscopy

For the detection of macropinocytosis uptake, HepG2 cells co-treated with Dextran (1 mg/ml) and LipoDox (20-25 ug/ml) were randomly assigned to 4 groups: (1) untreated cell, which serves as the control group; (2) 37° C. treatment, in which the cells were incubated at the temperature of 37° for 30 minutes; (3) 42° C. treatment, in which the cells were incubated at the temperature of 42° C. for 30 minute; (4) mEHT treatment, in which the cells were exposed to mEHT for 30 minute. After treatment, the cells were fixed with 3.7% paraformadehyde and stained with 1 ug/ml Heochst 33342 for 5 min at RT. The cells were washed with 1X PBS and resuspended in the mounting buffer. The cells were then dropped on to slides and analyzed by the confocal laser scanning microscope (FV1000; Olympus).

Flow Cytometry Assay

The treated cells were washed twice with phosphate-buffered saline (PBS). The cells were analyzed by flow cytometry. The percentage of positive cells were determined by using an Accuri C6 flow cytometer (BD Biosciences, Le Pont de Claix, France).

Animal Experiment

The BALB/c mice were obtained from the National Science Council Animal Center, Taipei, Taiwan, and were used at between 6 and 8 weeks of age. On day zero, the right femoral areas of BALB/c mice were injected subcutaneously with 5×10⁵ CT26 tumor cells. On day 14 following injection, the mice received local hyperthermia treatment. The two-step hyperthermia treatment was used for investigating the hyperthermia effect of LipoDox uptake in the tumor tissue. In the first-step of hyperthermia treatment, the mice were injected intravenously with LipoDox at 3 mg/kg followed immediately by a hyperthermia of 41° C. of water bath for 45 min. After the first-step hyperthermia treatment, mice were freed back to their cages and rested for 2 hours. For the second-step hyperthermia treatment, the mice were divided into two groups: (1) water bath (WB) treatment, in which the tumor were heated to 42° C. by water bath and (2) mEHT treatment. Tumor implants in the right femoral area of BALB/c mice were placed in the parallel electric condenser of the heating circuit. The treatment groups were given a single shot of mEHT for 30 min at a mean power of 1.5 W under 100 mg/kg Ketamine and 10 mg/kg Xylazine anesthesia. Intratumoral temperature was maintained at ˜42° C. on the treated side of each mice, as measured using optical sensors (Luxtron FOT Lab Kit, LumaSense Technologies, Inc., California, USA). The subcutaneous temperature underneath the electrode was maintained at 38˜40° C.

Quantification of Intratumoral Dox Accumulation

Tumor tissues were collected 15 min after the second hyperthermia of 42° C. for 30 min. Dox concentration in tumor tissues was quantified by fluorescence spectrometry. Briefly, the tumors tissues were dissected and then homogenized in acidified isopropanol and 0.025% triton X-100 and incubated at 4° C. for 24 hours. The fluorescence of Dox (Ex=472, Em=590) in the supernatant was measured.

### Example 1

### AM Radiofrequency Radiation Increases the Uptake of Liposome-Encapsulated Drug by Cells

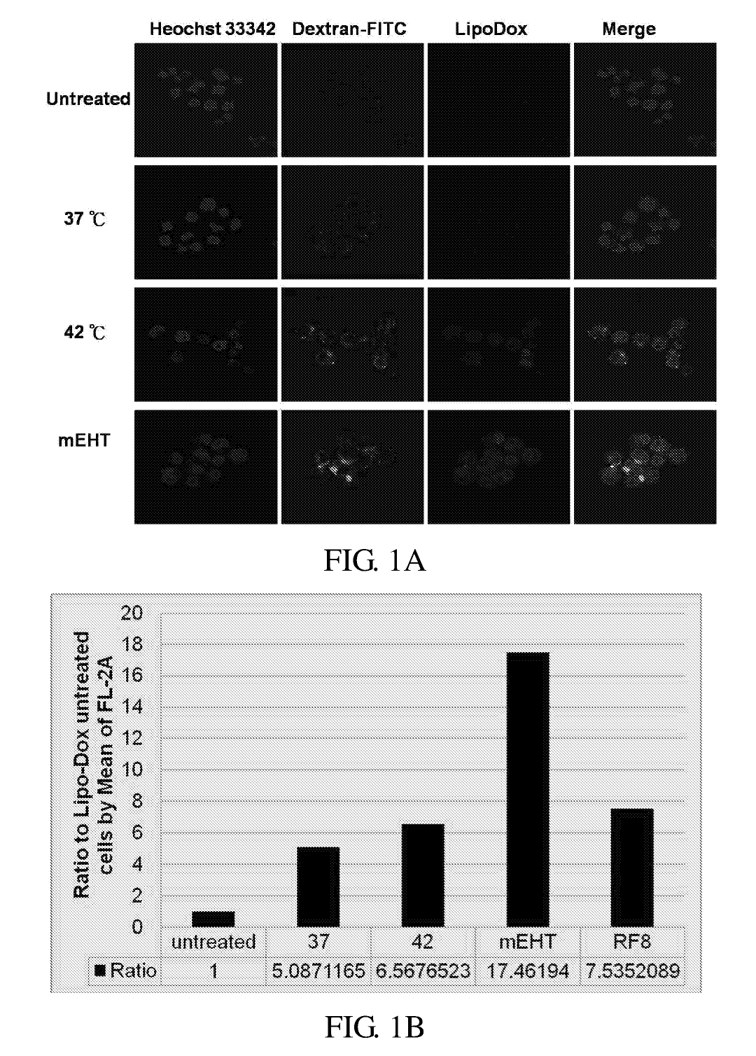

In this example, the effect of AM radiofrequency radiation on enhancing the absorption or uptake of liposome-encapsulated drug (i.e., lipo-dox) was investigated. The HepG2 cells were first treated with lipo-dox, then with AM radiofrequency radiation as described in Materials and Methods. Results were analyzed by flow cytometry and confocal microscopy, and depicted in FIGS. 1A-1B.

All the treatment groups (i.e., 37° C., 42° C. or mEHT treatment group) exhibited enhanced absorption of lipo-dox, as compared to that of the control group. It is noted that the fluorescence signal of the cell treated with mEHT was higher than that of the cell treated with 37° treatment or 42° treatment (data not shown). During the mEHT treatment, the temperature of the cells was maintained at 37° C. or 42° C., and the enhancement effect of mEHT was observed at both temperatures. As to the enhancement of the absorption of dox without liposome encapsulation, there was no obvious difference among the tested treatments. That data suggested that compared with the 37° C. or the 42° C. treatment, mEHT is advantageous in enhancing the absorption of the “liposome-encapsulated” drug. The confocal images of FIG. 1A confirmed the results.

FIG. 1B depicts the relative retention time of lipo-dox in the cells respectively treated with specified treatments (i.e., no treatment, 37° C. treatment, 42° C. treatment, mEHT treatment or RF8 treatment). The analysis result indicated that compared with the control group, 37° C., 42° C., mEHT and RF8 treatments respectively increased the retention of lipo-dox in the cells by about 5.09-, 6.57-, 17.46- and 7.54-fold.

These data demonstrated that compared with other treatments (including 37° C., 42° C. and RF8 treatments), mEHT more efficiently enhanced the uptake of liposome-encapsulated drug (e.g., lipo-dox).

### Example 2

### Mechanisms Involved in the Enhancement Effect or mEHT

It is known that cells may uptake particles of various sizes via various pathways; for example, the particles having the diameters of 0.5-55 μm, 50 nm, and 100 nm would be absorbed by the cells respectively through the macropinocytosis, Clathrin-mediated endocytosis, and Caveolae-mediated endocytosis pathways.

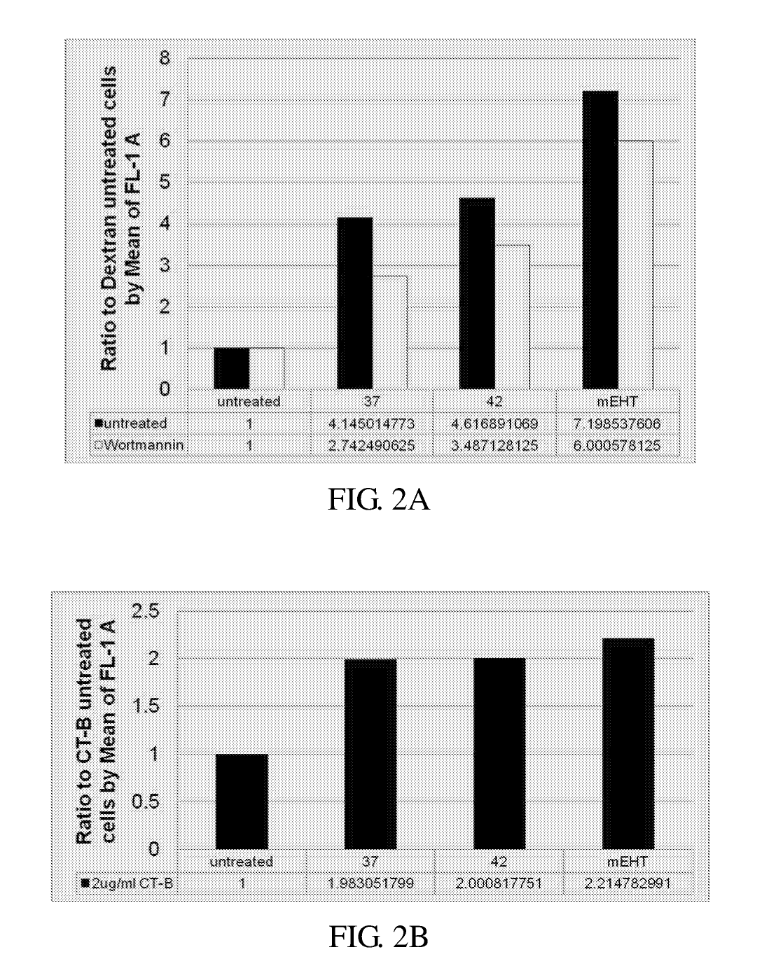

In this example, the HepG2 cells were first treated with dextran (about 0.5-5 μm), CT-B (about 100 nm), or transferrin (about 50 nm), the results were analyzed by flow cytometry assay so as to elucidate the possible mechanism involved in the enhancement effect of mEHT observed in Example 1. Results are depicted in FIGS. 2A-2C.

As illustrated in FIG. 2A, compared with that of the control group (i.e., untreated cell), all the treatment groups (i.e., 37° C., 42° C. and mEHT treatments) exhibited an enhanced absorption of dextran to the cells, in which the cells treated with mEHT emitted highest fluorescence signals. By contrast, administration of wortmannin inhibited the absorption of dextran.

The data of FIGS. 2B and 2C are similar with that of FIG. 1B, in which compared with the control group (i.e., untreated cell), all the treatment groups (i.e., 37° C., 42° C. and mEHT treatments) exhibited enhanced absorption of CT-B (FIGS. 2B) or transferrin (FIGS. 2C) by the cell.

Taken together, these data suggested that mEHT could enhance the absorption of a particle having a diameter from about 50 nm to 5,000 nm to the cells via the macropinocytosis, the Clathrin-mediated endocytosis, or the Caveolae-mediated endocytosis pathway.

### Example 3

### mEHT Enhanced Lipo-Dox Absorption In Vivo

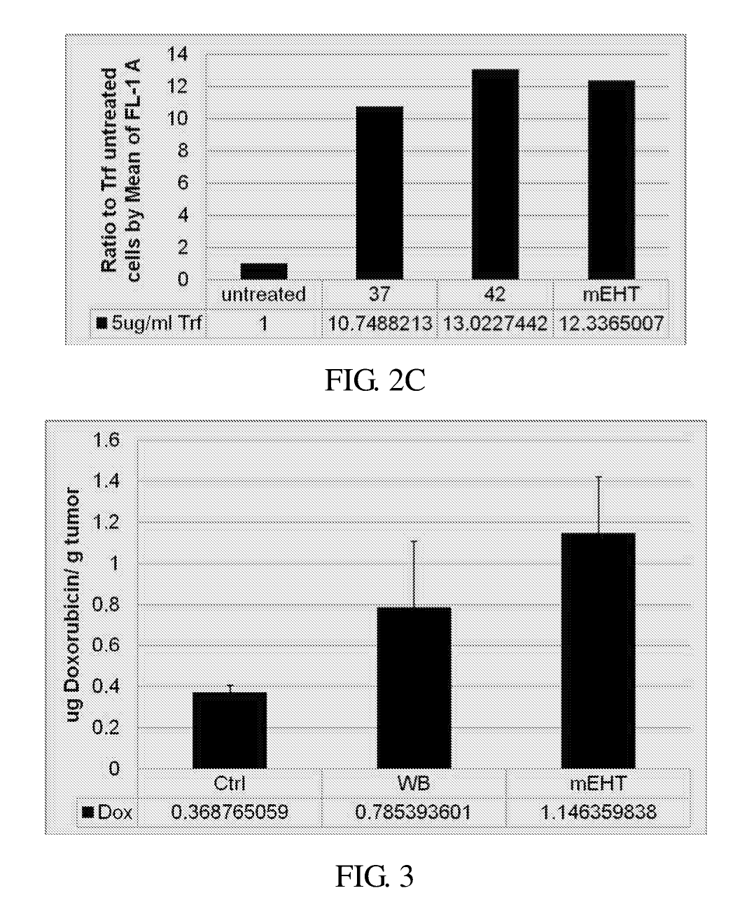

In this example, the effect of mEHT on the absorption of drugs was evaluated in the animal model. The data of FIG. 3 illustrate that the level of doxorubicin in the tumor of mice treated with mEHT is higher than that of the water bath (WB) treated mince and the control mice.

In conclusion, the present disclosure provides a method for efficiently improving the absorption of liposome-encapsulated drug by the tumor cell through applying an AM radiofrequency radiation to the tumor. Accordingly, the present disclosure confers a safely and effectively therapeutic effect on a subject having solid tumor.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A method of treating a subject having or suspected of having a solid tumor comprising:
(1) administering to the subject an effective amount of a liposome-encapsulated therapeutic agent; and
(2) applying an amplitude-modulated radiofrequency radiation to the solid tumor so as to enhance the absorption of the liposome-encapsulated therapeutic agent into the solid tumor, wherein the radiofrequency of the amplitude-modulated radiofrequency radiation is about 1-50 MHz.

2. The method of claim 1, wherein the radiofrequency of the amplitude-modulated radiofrequency radiation is about 5-45 MHz.

3. The method of claim 2, wherein the radiofrequency of the amplitude-modulated radiofrequency radiation is about 8-20 MHz.

4. The method of claim 1, wherein the power of the amplitude-modulated radiofrequency radiation is about 0.5-300 watts.

5. The method of claim 4, wherein the power of the amplitude-modulated radiofrequency radiation is about 25-150 watts.

6. The method of claim 4, wherein the power of the amplitude-modulated radiofrequency radiation is about 5-20 watts.

7. The method of claim 1, wherein the therapeutic agent encapsulated in the liposome is selected from the group consisting of, an anti-tumor agent, an anti-inflammatory agent, an anti-angiogenic agent, an immunomodulatory agent, and a radionuclide.

8. The method of claim 7, wherein the therapeutic agent is the anti-tumor agent, which is selected from the group consisting of bleomycin, epirubicin, estramustine, etoposide, 5-fluorouracil, doxorubicin, mitomycin C, cisplatin, paclitaxel, camptothecin, vincristine, vinblastine, methotrexate, mitoxantrone, thalidomide and squalamine.

9. The method of claim 8, wherein the anti-tumor agent is bleomycin, doxorubicin, mitomycin C, cisplatin or paclitaxel.

10. The method of claim 7, wherein the therapeutic agent encapsulated in the liposome has a concentration of about 5-800 ug/ml.

11. The method of claim 1, wherein the liposome has a diameter ranging from about 50-5,000 nm.

12. The method of claim 1, wherein the solid tumor is melanoma, esophageal carcinoma, gastric carcinoma, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, cervical cancer, or head and neck squamous cell carcinoma.

13. The method of claim 1, wherein the subject is a human.

14. The method of claim 1, wherein the liposome-encapsulated therapeutic agent is administered to the subject via an oral, enteral, nasal, topical, transmucosal, intramuscular, intravenous, intraarterial, subcutaneous, intraperitoneal, or intratumoral route.

15-42. (canceled)
